(19) United States
(12) Patent Application Publication
(10) Pub. No.: US 2023/0019348 A1
(43) Pub. Date: Jan. 19, 2023

(54) Bone Cement Mixing System and Apparatus

(71) Applicants: Christopher Pedicini, Brentwood, TN (US); Joshua Pedicini, Nashville, TN (US)

(72) Inventors: Christopher Pedicini, Brentwood, TN (US); Joshua Pedicini, Nashville, TN (US)

(21) Appl. No.: 17/674,759

(22) Filed: Feb. 17, 2022

Related U.S. Application Data
(63) Continuation-in-part of application No. 17/125,247, filed on Dec. 17, 2020.
(60) Provisional application No. 63/150,542, filed on Feb. 17, 2021.

(51) Int. Cl. A61B 17/88

(52) U.S. Cl. CPC A61B 17/8833; A61B 2017/8838; A61B 2017/00876

(57) ABSTRACT

An apparatus for mixing bone cement for use in orthopedic surgeries includes a mixing region and an agitator for mixing the cement ingredients. The apparatus can be operated or acted on by a power tool for the mixing of the bone cement. The agitator may act as a generator to charge embedded electronics of the apparatus such as with a magnet in the agitator that interacts with a wire coil outside of the agitator. A timer of the apparatus may be started by a sensor of the apparatus, in response to a mechanical torque value, or by a magnetic sensor that can sense a start state of the apparatus. When the timer is complete, a cue by the apparatus signals to the user that the mixed cement is ready and able to be applied.

### CROSS-REFERENCE TO RELATED APPLICATIONS

The present disclosure claims priority under 35 U.S.C. § 119 on pending U.S. Provisional Application Ser. No. 63/150,542 filed on Feb. 17, 2021, the disclosure of which is incorporated by reference. The present disclosure is also a continuation-in-part of and claims priority under 35 U.S.C. § 120 on pending U.S. patent application Ser. No. 17/125,247 filed on Dec. 17, 2020, the disclosure of which is incorporated by reference.

### FIELD OF DISCLOSURE

The present disclosure relates to the preparation of adhesive compounds for use in orthopedic surgeries, and more specifically, to the mixing of an at least two-component bone cement for use in implant fixation in surgeries, such as (but not necessarily limited to) Total Hip Arthroplasty and Total Knee Arthroplasty.

### BACKGROUND OF THE DISCLOSURE

This disclosure relates to an improvement of bone cement mixing techniques and systems. Bone cement is used in the field of surgery to assist in fixing a surgical implant to a patient's bone, for example.

In general, polymethyl methacrylate (PMMA) is the most commonly found bone cement in orthopedic and trauma surgery. The cement product is formed by mixing a liquid methacrylate (MMA) monomer and a powder MMA co-polymer (it being understood that this liquid and powder also contains other factors such as accelerators and initiators that enable adequate polymerization). This mixing is only done in the operating room when, in the example of orthopedic knee or hip replacement surgery, the implant is ready to be fixed in the bone. Thus, proper preparation of the cement occurs under very constrained conditions, creating a need for precision in the mixing process.

Most bone cement mixing systems currently in use rely on a surgeon's assistant to manually mix the bone cement powder and liquid monomer. After this manual mixing is done, the surgeon's assistant is required to perform a qualitative test (commonly called the “fingertip test”) to determine when the bone cement is ready to be applied. This test is imprecise at best and further suffers from subjectivity and variance from the fact that surgeons often have multiple different assistants who prepare the bone cement, each of whom may have a different technique/mixing speed that results in differing “ready” times for the bone cement. Automated mixing systems exist in the prior art but they too are subject to the qualitative and unreliable “fingertip test” that must be performed manually. In addition, different types of gloves have been shown to result in “ready” times varying by as much as 250%. This can be related to surgeon feel, glove thickness, glove material and glove surface, amongst other things.

Depending on the surgeon's particular assistant, the prepared bone cement may be ready for use from anywhere between two to eight minutes after mixing is completed. The bone cement only stays in its “ready” state for about three minutes (which ready state is also known as “working time” or “application time”), and if the surgeon does not have the bone and implant prepared in that amount of time then the bone cement hardens and can no longer be used. Alternatively, a situation can arise where the bone and implant are ready, and the cement is still in its “waiting” phase. The waiting phase is similarly undesirable for at least the reason that surgery time is expensive, and efficiency is of utmost important in the operating room.

All of the currently available systems and methods for bone cement mixing suffer from one or more of the following disadvantages: ambiguous and imprecise indicator of when the mix is ready (from the fingertip test), cement nonuniformity, mixing techniques differing and dependent on the particular different surgeon's assistant that is performing the mixing at any one time (which can result in differing and unpredictable ready/preparation times), and the fact that the process is time-consuming in and of itself.

### SUMMARY OF THE DISCLOSURE

In view of the foregoing disadvantages inherent in the prior art, the purpose of the present disclosure is to provide a bone cement mixing system and apparatus that provide a uniform mix quickly and with clear indicators of when the mix is ready for use and when the mix is no longer useable. It is understood that PMMA and bone cement refer to the same compound, however, it should be apparent that the apparatus described herein may be used with other multicomponent adhesive materials and compounds used in orthopedic surgery.

In an embodiment, a mixing apparatus uses an asymmetrical mixing pattern to achieve a more uniform mix. In an embodiment the apparatus comprises a mixing region (such as a reservoir, container or bowl, for example) in which the cement components may be disposed for the mixing process and an agitator further disposed for mixing said cement components

In an embodiment, a mixing apparatus includes a temperature sensing means (also referred to herein as temperature indicator) such as a thermocouple. In an embodiment, a cue (audible, visual, or other such signal) indicates that the mixed cement is no longer pliable enough to be used.

In an embodiment, the mixing apparatus can be operated by a power tool for the mixing of the bone cement. That is, the power tool can be operatively coupled to the agitator and the agitator so attached or coupled to the power tool such that, when the power tool operates, the power tool causes the agitator to rotate or otherwise move within the mixing region.

In an embodiment, the power tool and/or mixing apparatus has a mechanical torque limiter between an input and agitator that may slip, overrun, break or otherwise inhibit the agitator as a result of mixing torque exceeding a threshold value. The mechanical torque limiter can provide a cue (audible, visual, or other) indicating that the mixing phase is complete.

In an embodiment, the apparatus comprises a timer. In another embodiment, the timer of the apparatus may be started by a sensor of the apparatus that determines that the mechanical torque limit has been reached. When the timer is complete, a cue (such as an audible, visual, or other cue) by the apparatus signals to the user that the mixed cement is ready and able to be applied.

In an embodiment, the unmixed bone cement powder and liquid are compartmentalized within the mixing apparatus. In a further embodiment, the inside of the mixing apparatus remains completely sealed and sterile until the bone cement has been mixed and the user is ready to apply said bone cement, at which point an opening in the apparatus is exposed for cement collection.

In another embodiment, a power tool for use with a mixing apparatus is provided. The tool includes a torque limiter that may cease agitation of a mix in the apparatus by the tool once a threshold torque is met. In an embodiment, the torque limiter indicates to the user that a threshold torque has been reached.

### DESCRIPTION OF THE DRAWINGS

The advantages and features of the present disclosure will become better understood with reference to the following detailed description and claims taken in conjunction with the accompanying drawings, wherein like elements are identified with like symbols.

FIG. 1 shows a cross sectional view of a mixing apparatus in accordance with an exemplary embodiment of the present disclosure;

FIG. 2 shows a mixing apparatus wherein cement components are contained within the apparatus prior to mixing in accordance with an exemplary embodiment of the present disclosure.

FIG. 3 shows a mixing apparatus and power tool in accordance with an exemplary embodiment of the present disclosure.

FIG. 4 shows the shearing of a mechanical torque limiting element between an input and the agitator of a mixing apparatus, in accordance with an exemplary embodiment of the present disclosure.

FIG. 5 shows a across sectional view of a mixing apparatus in accordance with another exemplary embodiment of the present disclosure.

### DETAILED DESCRIPTION OF THE DISCLOSURE

The exemplary embodiments described herein detail for illustrative purposes are subject to many variations in structure and design. That is, it is understood that various omissions and substitutions of equivalents are contemplated as circumstances may suggest or render expedient, but these are intended to cover the application or implementation without departing from the spirit or scope of the claims of the present disclosure. The terms “first,” “second,” and the like, herein do not denote any order, quantity, or importance, but rather are used to distinguish one element from another, and the terms “a” and “an” herein do not denote a limitation of quantity, but rather denote the presence of at least one of the referenced item.

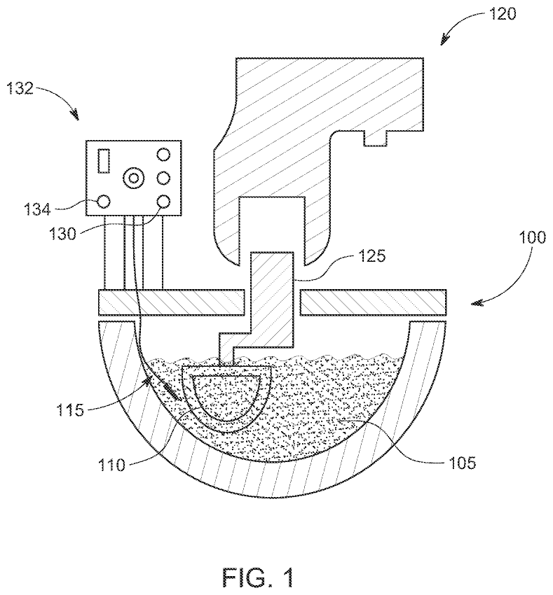

The present disclosure provides for an apparatus 100 for mixing at least two-component cement as shown in FIG. 1, for example, which cement is for use in orthopedic surgeries. In an embodiment the apparatus 100 comprises a mixing region 105 (such as a reservoir, container or bowl, for example) in which the cement components may be disposed for the mixing process. The apparatus 100 comprises an agitator 110 which agitator actuates to mix the cement ingredients (such as, for example, a powdered cement polymer and a liquid monomer) together for forming the cement. In an embodiment, the agitator 110 comprises an impeller blade, however, it will be apparent that the agitator 110 may comprise a whisk, screw, paddle or any other configuration that facilitates mixing of the elements of the cement. In an embodiment, the mixing apparatus 100 uses an asymmetrical mixing pattern to achieve a more uniform mix of a cement product.

Referring again to FIG. 1, in an embodiment, the mixing apparatus 100 includes a temperature sensing means 115 (also referred to herein as temperature indicator or temperature sensor) such as a thermocouple. In an embodiment, a cue (audible, visual, or other such signal) indicates that the cement mix can no longer be used. This cue can come from a circuit board (such as a circuit board of the apparatus 100, for example) that reads the temperature from the temperature sensor 115. In an embodiment the temperature indicator comprises an LED panel that alights when the temperature sensing means 115 reads a temperature at which the cement mix can no longer be used. In an embodiment, the temperature indicator 115 can be a temperature dot or dots disposed on (and therefore easily viewable) the mixing apparatus 100. In a further embodiment, the temperature dot panel may indicate when the mix is approaching being too unpliable to use. In a still further embodiment, the temperature sensing means is integral to the mixing apparatus and further, part of or all of the apparatus can change color with changes in temperature (such as in an embodiment wherein the apparatus comprises a thermochromic material or component.) It should be understood that the behavior of cement is temperature-sensitive such that when the cement's temperature is increased, the working time of the mixture will be comparatively reduced. In an embodiment, the temperature indicator references ambient temperature.

In an embodiment, the temperature indicator 115 is a thermochromic strip. In an embodiment, said thermochromic strip may be predisposed on or in the apparatus 100 to provide a color indication of the temperature of the cement.

In an embodiment, the mixing apparatus 100 has embedded electronics 132 that include buttons, switches, LED(s), a circuit board, and/or microcontroller(s). It will be apparent that such embedded electronics may control and/or be utilized to operate the apparatus 100.

In an embodiment and as shown in FIG. 1, the mixing apparatus 100 can be operated or acted on by a power tool 120 for the mixing of the bone cement. That is, the power tool 120 can be operatively coupled to the agitator 110 and the agitator 110 so attached or coupled to the power tool 120 such that, when the power tool 120 operates (such as in the case of a rotary motion end (e.g. a motorized drill or screwdriver)), the power tool 120 causes the agitator 110 to rotate or otherwise move within the mixing region 105.

In said embodiment, the power tool 120 and/or mixing apparatus 100 has a mechanical torque limiter 125 between an input of the tool 120 and/or apparatus 100 and the agitator 110 that slips, overruns, breaks, indicates or otherwise inhibits the agitator 110 as a result of mixing torque exceeding a threshold value. That is, once the bone cement powder and monomer are in contact with one another, a polymerization reaction occurs and, as a result, the mixture's viscosity starts to increase. This viscosity increase requires an increasing in mixing torque. A threshold torque value is reached at the viscosity that indicates ideal application time of the bone cement. Upon reaching this threshold value, the mechanical torque limiter 125 can provide a cue (audible, visual, or other) indicating that the mixing phase is complete. Such mechanical torque limiter 125 can operate by means of a slip-clutch, for example.

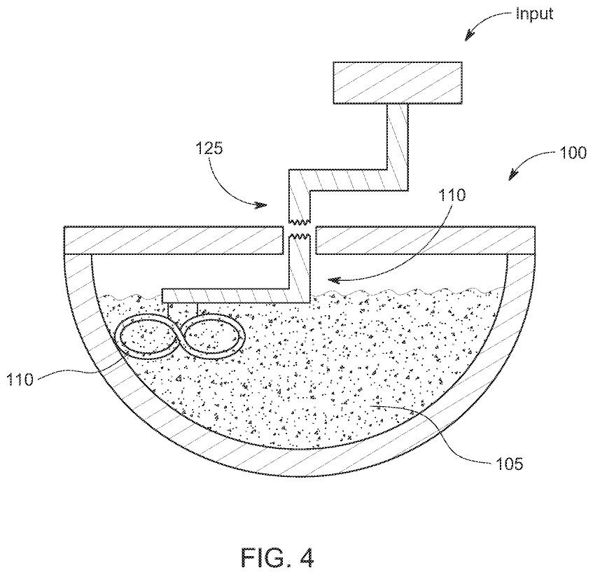

In an embodiment, the apparatus 100 comprises a timer 130, which timer may be incorporated as part of a microcontroller, for example. In an embodiment, the timer 130 of the apparatus 100 may be started by a sensor 134 of the apparatus 100 or by the user in response to a mechanical torque value, for example. When the timer 130 is complete, a cue (such as an audible, visual, or other cue) by the apparatus 100 signals to the user that the mixed cement is ready and able to be applied. In an embodiment the initial countdown setting is controlled by the temperature of the mixed cement. The timer 130 improves outcomes by informing the surgeon and any assistants as to exactly how much time they have until the bone cement is no longer in a state where it is able to be applied. This allows for greater predictability and efficiency in the operating room. In an embodiment, the timer countdown is displayed digitally on the mixing apparatus 100. In a still further embodiment, the mixing apparatus' 100 embedded electronics recognize when the mechanical torque limit has been reached and starts the timer 130. In an embodiment, the mixing apparatus 100 with mechanical torque limiter 125 is manually operated by the user. In a further embodiment, the mechanical torque limiter 125 prevents overmixing by not allowing further mixing after a threshold torque has been reached. This may occur, for example, in the event of an intentional shearing of the connection between the input and the impeller/agitator 110, as shown in FIG. 4 for example.

In an embodiment, the bone cement is ready for use immediately or nearly immediately after mixing is completed. This can be accomplished by configuring the mixing apparatus 100 to only stop mixing when the cement is ready to be applied (i.e., such as when a viscosity of the mixture is reached that indicates ideal application time of the bone cement, such time sometimes commonly referred to as working time).

In an embodiment, the mixing apparatus comprises a means for testing the tackiness of the mix. This could, for example, be a mechanical “finger” that constantly dips into the cement mix measuring the tack of the mixture. Said tackiness measuring means can indicate to the surgeon's assistant/surgeon that the mix is in its dough point and is ready to be applied.

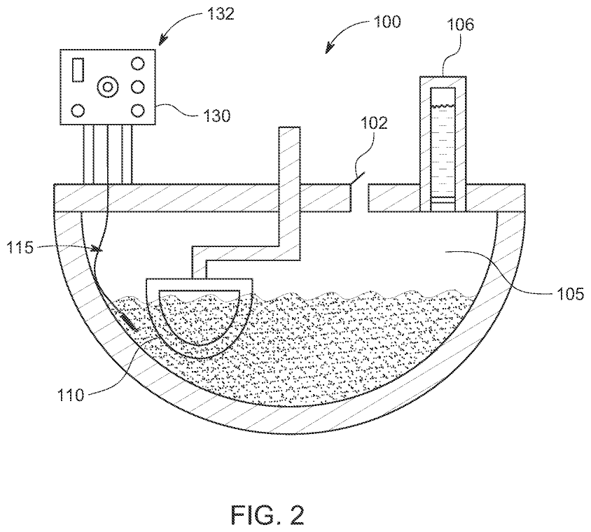

In another embodiment, the unmixed bone cement powder and liquid are contained within the mixing apparatus 100. Prior to mixing, the powder and liquid are preferably contained apart from one another. For example, as shown in FIG. 2, the powder can be sealed within the mixing region 105 (such as the reservoir, for example) of the apparatus 100 while the liquid is contained in a separate compartment 106 of the mixing apparatus 100, which separate compartment 106 is in fluidic communication with the mixing region 105. When the user is ready, he can dispense the liquid into the mixing region 105 (through a selectively openable valve or port that is in communication with the mixing region 105, the opening/closing controls for which may be contained in the apparatus' 100 embedded electronics, for example) in physical proximity to the powder and begin mixing. This allows for clean, sterile, and streamlined mixing.

In a further embodiment, the inside of the mixing apparatus 100 remains completely sealed and sterile until the bone cement has been mixed, with, for example, the liquid being maintained in a sterile condition prior to the dispensing of the liquid into the mixing region 105. This configuration provides the same benefits as mixing under vacuum but eliminates the need for a hose and vacuum pump. In an embodiment, the mixing apparatus 100 contains a one-way valve 102 that prevents pressure buildup when mixing in a sealed environment mixing region 105.

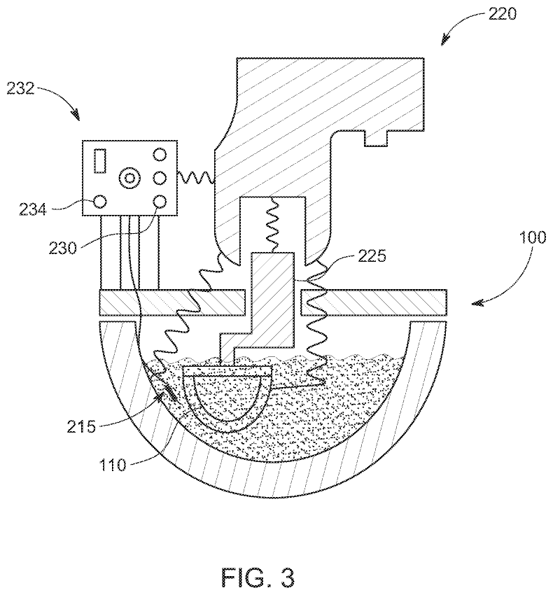

In another embodiment, and as shown in FIG. 3 a power tool 220 for use with a mixing apparatus is provided. The tool is capable of operatively coupling with a mixing apparatus to agitate the contents of the mixing apparatus, such as by having the tool attach to an impeller or other agitating means 110 of the mixing apparatus. The power tool 220 comprise a torque limiter 225 as elsewhere described herein, which torque limiter slips, overruns, breaks or otherwise inhibits the power tool 220 as a result of mixing torque exceeding a threshold value.

In an embodiment, the tool 220 comprises a timer 230, which timer may be incorporated as part of a microcontroller of the tool. In an embodiment, the user presses a button on the power tool 220 after the mechanical torque limit has been reached, which button press may start a timer 230 on the microcontroller. In another embodiment, the timer 230 of the tool 220 may be started by a sensor 234 of the tool 220 that determines that the mechanical torque limit has been reached. When the timer 230 is complete, a cue (such as an audible, visual, or other cue) by the tool 220 signals to the user that the mixing process has completed or reached a particular stage.

In an embodiment, the tool 220 has embedded electronics 232 that include buttons, switches, LED(s), a circuit board, and/or microcontroller(s). It will be apparent that such embedded electronics may control and/or be utilized to operate the tool 220.

In an embodiment, the tool 220 includes a temperature sensing means 215 (also referred to herein as temperature indicator) such as a thermocouple, which temperature sensing means may operatively couple to a mixing apparatus to sense temperature of or within the mixing apparatus. In an embodiment, a cue (audible, visual, or other such signal) indicates that a certain temperature has been reached in the mixing apparatus. This cue can come from a circuit board (such as a circuit board of the tool 220, for example) that reads the temperature from the temperature indicator 215. In an embodiment, the temperature indicator 215 can be a temperature dot or dots disposed on (and therefore easily viewable) on the tool 220. In an embodiment the temperature indicator comprises an LED panel that alights when the temperature sensing means 215 reads a temperature at which the cement mix cannot be used any longer. In a further embodiment, the temperature dot panel may indicate when a mix in the apparatus is approaching being too unpliable to use.

In an embodiment, the mechanical torque limiter may cause a decoupling of the agitator when a particular torque limit is met. In an exemplary embodiment, and as shown in FIG. 4, the coupling between an input (such as a power tool or a handle otherwise coupled to the agitator) and the agitator shears when a torque limit is reached. This embodiment has an advantage of providing an easily discernable visual, audible and/or physical cue that the cement has reached the condition in which it is in its working state.

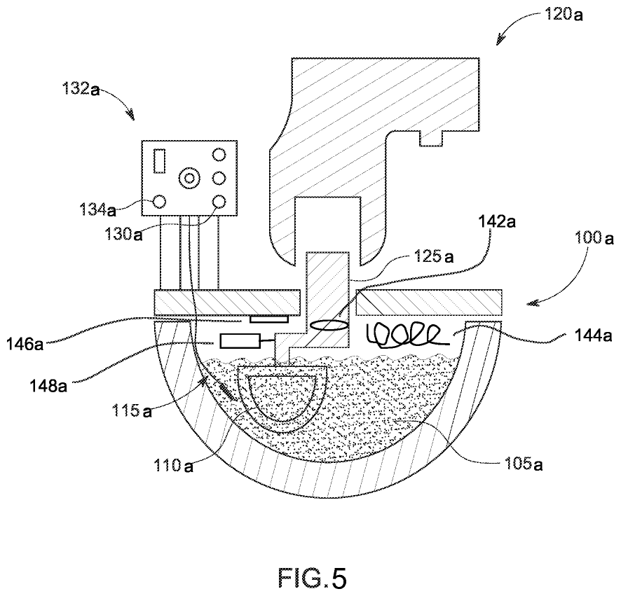

In another embodiment, and referring now to FIG. 5, a mixing apparatus 100a comprises a mixing region 105a (such as a reservoir, container or bowl, for example) in which the cement components may be disposed for the mixing process. The apparatus 100a comprises an agitator 110a which agitator actuates to mix the cement ingredients (such as, for example, a powdered cement polymer and a liquid monomer) together for forming the cement. In an embodiment, the agitator 110a comprises an impeller blade, however, it will be apparent that the agitator 110a may comprise a whisk, screw, paddle or any other configuration that facilitates mixing of the elements of the cement. In an embodiment, apparatus 100a comprises a temperature sensing means 115a similar in configuration to temperature sensing means 115 described elsewhere herein.

In an embodiment, the mixing apparatus 100a has embedded electronics 132a similar in configuration to electronics 132 described elsewhere herein. In an embodiment and as shown in FIG. 5, the mixing apparatus 100a can be operated or acted on by a power tool 120a for the mixing of the bone cement through operative coupling with agitator 110a similar to the configuration of power tool 120 as described elsewhere herein. Power tool 120a and/or mixing apparatus 100a may comprise a mechanical torque limiter 125a between an input of the tool 120a and/or apparatus 100a and the agitator 110a that slips, overruns, breaks, indicates or otherwise inhibits the agitator 110a as a result of mixing torque exceeding a threshold value, similar in configuration to the torque limiter 125 disclosed elsewhere herein. In an embodiment, the apparatus 100a comprises a timer 130a, similar to the timer 130 described elsewhere herein, which timer 130a may be started by a sensor 134a that is operatively coupled to the timer 130a.

In an embodiment, the mixing apparatus 100a uses an asymmetrical mixing pattern to achieve a more uniform mix. In said embodiment, the agitator may comprise two paddles at ends of the agitator that are opposite to one another, wherein one paddle end scrapes the walls of the mixing region 105a and the other paddle end is spatially offset from the walls of the mixing region 105a walls so that agitator 110a may compresses the cement against the wall.

In an embodiment, mixing apparatus 100a mixing apparatus may comprise thermochromic ink (such as a mixing region 105a that is molded from thermochromic ink). This may cause the mixing apparatus 100a to change color for example from transparent and clear (to indicate that the mixed cement is ready for use)or from to opaque and red (to indicate that the cement should no longer be used).

In an exemplary embodiment, embedded electronics 132a of mixing apparatus 100a may comprise a microcontroller and audible or visual indicator. In an embodiment, said embedded electronics are powered by an energy storage means 148a such as a battery, capacitor, supercapacitor, and the like. The mixing apparatus 100a can be configured such that the embedded electronics 132a are not powered until the mixing process has started. For example, if a battery is used, the battery may power the electronics at the initiation of mixing. This may be accomplished by providing a tab of insulating material predisposed between a terminal of the battery and a circuit of the embedded electronics circuit for example, with said tab being removed by the initiation of the mixing process (e.g. the rotation of the agitator 110a).

In a further embodiment, the rotation of the agitator 110a can act as a generator to charge the energy storage means 148a such that the energy storage means may power the embedded electronics 148a. In an exemplary embodiment, a rotatable portion of the agitator 110a may comprise at least one magnet (such as magnet 142a shown in FIG. 5) that is perpendicular to a stationary portion of the mixing apparatus, which stationary portion can comprise at least one coil of wire 144a. During actuation of the agitator 110a, said at least one embedded magnet 142a can induce a current in said stationary coil of wire 144a, which current may charge a supercapacitor, rechargeable battery or other energy storage means 148a to power the embedded electronics. In an embodiment, the microcontroller of embedded electronics 132a may, upon powering on, (for example, by means of the aforementioned energy storage means) starts a timer 130a once the agitator 110a has stopped rotating (which stopping indicates that the mixing process has finished).

In an embodiment, the power tool 120a has a mechanical torque limiter 125a between an actuator and impeller that slips, overruns, breaks, or otherwise inhibits the agitator 110a as a result of mixing torque. In a further embodiment, the mixing apparatus' embedded electronics 132a recognizes when the mechanical torque limit has been reached and starts the timer 130a. In said embodiment, a rotatable portion of the agitator 110a can include at least one magnet 142a while a stationary portion of the mixing apparatus can contain at least one magnetic sensor 146a (such as a hall sensor for example), wherein said at least one magnetic sensor 146a can input to a microcontroller of the embedded electronics 132a and indicate the start state and end state of the mixing process (such as a start state that is only recognized after the mixing apparatus agitator 110a and/or impeller has been rotated and then ceases to rotate. This could occur, for example, any time between one and sixty seconds after mixing has been started. Once said start state is recognized, said end state is recognized when the mixing apparatus actuator and/or input of power tool 120a has stopped rotating (such as, for example, when the mechanical torque 125a limiter breaks). When the microcontroller has recognized said end state, the microcontroller can start a countdown by the timer 130a. At the end of the timer countdown, in an embodiment, the cement is ready to be used.

The foregoing descriptions of specific embodiments of the present disclosure have been presented for purposes of illustration and description. They are not intended to be exhaustive or to limit the present disclosure to the precise forms disclosed, and obviously many modifications and variations are possible in light of the above teaching. The exemplary embodiment was chosen and described in order to best explain the principles of the present disclosure and its practical application, to thereby enable others skilled in the art to best utilize the disclosure and various embodiments with various modifications as are suited to the particular use contemplated.

What is claimed is:

1. An apparatus for mixing at least a two-component bone cement, the apparatus comprising
a mixing region,
an agitator, said agitator comprising at least one magnet,
a means for actuating the agitator rotationally,
an energy storage means,
wherein upon rotation of the agitator, the magnet of the agitator inductively charges said energy storage means, and
wherein the energy storage means may further power the apparatus.

2. The apparatus of claim 1, wherein the means for actuating the agitator comprises a power tool.

3. The apparatus of claim 1, wherein the agitator comprises two paddles disposed on opposite sides of the agitator, and wherein a first paddle end engages a surface of the mixing region and a second paddle does not engage a surface of the mixing region.

4. The apparatus of claim 1, wherein the apparatus further comprises a torque limiter, said torque limiter is disposed between said agitator and said means for actuating the actuator and wherein said agitator is no longer actuated when the torque limiter reaches a predetermined value.

5. The apparatus of claim 1, the apparatus further comprising a temperature sensing means, said temperature sensing means capable of sensing and signaling at least one of a cement mix in the mixing apparatus being ready for use, no longer ready for use, the temperature of a mixture in the mixing region of the apparatus.

6. The apparatus of claim 1, wherein the means for actuating the agitator further comprises a power tool.

7. The apparatus of claim 14, wherein the apparatus further comprises a torque sensor that is capable of sensing and signaling that a threshold torque of the apparatus has been reached.

8. The apparatus of claim 7, said apparatus further comprising a timer, and wherein said timer is initiated after said torque sensor has sensed that a threshold torque of the apparatus has been reached.

9. The apparatus of claim 1, wherein the apparatus further comprises embedded electronics that are powered by the energy storage means.

10. The apparatus of claim 1, wherein the apparatus further comprises a magnetic sensor and the at least one magnet of the agitator is communicatively coupled with the magnetic sensor.

11. The apparatus of claim 10, wherein the apparatus further comprises a microcontroller and wherein the magnetic sensor can input to the microcontroller to indicate at least one of a start state and an end state of the mixing by the apparatus.

12. The apparatus of claim 1, wherein the embedded electronics are not powered until the mixing by the apparatus has started.

13. As apparatus for mixing at least a two-component bone cement, the apparatus comprising
a mixing region,
an agitator, said agitator comprising at least one magnet,
a means for actuating the agitator rotationally,
an energy storage means, and
a control circuit,
wherein said control circuit is not enabled until the agitator begins rotating.

14. The apparatus of claim 13, wherein the agitator must complete at least 90 percent of one rotation before the energy storage means may power the control circuit.

15. The apparatus of claim 13, the apparatus further comprising a temperature sensing means, said temperature sensing means capable of sensing and signaling at least one of a cement mix in the mixing apparatus being ready for use, no longer ready for use, the temperature of a mixture in the mixing region of the apparatus.

16. The apparatus of claim 13, wherein the apparatus further comprising a torque sensor that is capable of sensing and signaling that a threshold torque of the apparatus has been reached.

17. The apparatus of claim 16, said apparatus further comprising a timer, and wherein said timer is initiated after said torque sensor has sensed that a threshold torque of the apparatus has been reached.
